(19) United States
(12) Patent Application Publication
(10) Pub. No.: US 2011/0104752 A1
(43) Pub. Date: May 5, 2011

(54) Variation of Recombinant Expression Titres By Optimising Bacterial Ribosome Binding Sites

(75) Inventors: Laurent Chevalet, Cuvat (FR); Kinsey Maundrell, Chene-Bougeries (CH)

(73) Assignee: Merck Serono S.A., Coinsins (CH)

(21) Appl. No.: 12/922,550

(22) PCT Filed: Mar. 13, 2009
(86) PCT No.: PCT/EP09/53024

§ 371 Date: Dec. 15, 2010

Related U.S. Application Data
(60) Provisional application No. 61/072,221, filed on Mar. 28, 2008.

(30) Foreign Application Priority Data
Mar. 14, 2008 (EP) 08152803.6

(51) Int. Cl. C12P 21/02; C40B 50/06; C07H 21/00; C07H 21/04; C12N 15/63; C12N 1/21

(52) U.S. Cl. 435/69.1; 506/26; 536/24.1; 435/320.1; 435/252.33

(57) ABSTRACT

The present invention provides a method for optimising the ribosome binding site of a promoter for the expression of a gene encoding a polypeptide of interest, placed under the control of said promoter. The invention also relates to a vector containing such optimised promoters, a prokaryotic host cell transformed by said vector, as well as a method for producing a recombinant protein of interest.

### FIELD OF THE INVENTION

The present invention provides a method for optimising gene expression. The invention also relates to optimised promoters, vectors containing such promoters, a prokaryotic host cell transformed by said vector, as well as a method for producing a recombinant protein of interest.

### BACKGROUND OF THE INVENTION

Bacterial expression systems generally make use of a host strain transformed with an episomal element (plasmid) able to drive the expression of a foreign gene. The strain provides the necessary biological functions for plasmid retention, replication and transmission, as well as transcription and translation of the gene of interest. The plasmid typically carries (i) an expression cassette comprising a promoter, the gene of interest and a transcription terminator, (ii) a marker gene which allows for selection of recombinant bacteria, and (iii) a replicon containing the origin of DNA replication together with its associated cis-acting elements. The classification of expression systems according to the promoter used relates to the first assumption that the quality of the promoter is the most important asset.

Escherichia coli (E. coli) is the primary bacterial species for the expression of heterologous proteins. It is well characterized genetically (Blattner et al., 1997), grows in relatively inexpensive media and expression is fast, typically producing peak yields of protein in 1-2 days. Plasmid vectors, strains with different genetic backgrounds (Terpe, 2006) and high-cell density cultivation processes are part of the toolkit that allows E. coli to meet a wide spectrum of research, development and commercial needs (Graumann and Premstaller, 2006).

Efficient protein expression in E. coli requires adaptation of expression system components to the gene of interest: regulated and tunable promoter, optimised codon usage of the gene of interest for E. coli, N- or C-terminal tags acting as solubilization partners, translational enhancers, purification and/or detection tags, and a leader sequence in cases where export to the periplasm is desirable (see Peti and Page (2007) for a review). However, even in such “optimised” conditions, it is not uncommon that genes of interest are poorly converted into protein product.

Initiation is the rate-limiting step of translation. Sequence patterns and structural motifs at the mRNA level influence the transition of ribosome-initiator tRNA-mRNA ternary complexes into an initiation-competent ribosome able to proceed along the message and to perform polypeptide elongation. Investigations on the elements affecting translation in prokaryotes have pointed to a number of key determinants (reviewed by McCarthy and Brimacombe, 1994).

The ribosome binding site (RBS) is a sequence of the mRNA at which assembly of the 30S and 50S subunits takes place. One key determinant of the RBS is the Shine-Dalgarno (SD) sequence (Shine and Dalgarno, 1974). The SD element acts by base pairing with an anti-SD sequence (5′-CCUCCUUA-3′) near the 3′ end of the 16S rRNA. A statistical analysis of bacterial mRNAs reveals that the sequence of this region is not random (Gold, 1988), suggesting that it drives the 30S subunit to distinguish between true RBS and <<RBS-like>> sequences. One key determinant of the RBS is the Shine-Dalgarno (SD) sequence (Shine and Dalgarno, 1974). The SD element acts by base pairing with an anti-SD sequence (5′-CCUCCUUA-3′) near the 3′ end of the 16S rRNA. The importance of this interaction is supported by the strong representation of purines in the [−12;−7] region of natural RBS of E. coli mRNAs. This bias is confirmed in a population of 158 sequences selected from a library of randomised RBS for their capacity to promote the expression of a reporter gene (Barrick et al., 1994).

In addition to the SD element, other nearby sequences or structures contribute to the overall efficiency of translation initiation, and these motifs include the nature of the start codon (Ringquist et al., 1992), the distance between the SD sequence and the start codon (Chen et al., 1994a), the degree of secondary structures at the vicinity of the SD sequence (De Smit and van Duin, 1994; De Smit and van Duin, 1990), the presence of adenine-rich motifs within mRNA coding sequences (Chen et al., 1994b), as well as several upstream pyrimidine-rich (Boni et al., 1991; Komarova et al., 2005; Tzareva et al., 1994) and AU-rich sequences (Komarova et al., 2005), and downstream A-rich (Chen et al., 1994b; Brock et al., 2007) and CA-rich (Martin-Farmer and Janssen, 1999) tracts. Non linear analyses applied to the translational activities measured in biological experiments have led to the definition of general rules for predicting translational efficiency that, to some extent, confirm previously reported traits (Mori et al., 2006).

The creation of a RBS driving high-level expression of a foreign gene involves optimising the complex relationships between the various elements mentioned above and the following publications are representative of the studies addressing this issue.

In Barrick et al. (1994), 185 clones with randomised ribosome binding sites, from position −11 to 0 preceding the coding region of beta-galactosidase, were selected and sequenced. The translational yield of each clone was determined; they varied by more than 3000-fold. Multiple linear regression analysis was used to determine the contribution to translation initiation activity of each base at each position. Features known to be important for translation initiation, such as the initiation codon, the Shine/Dalgarno sequence, the identity of the base at position −3 and the occurrence of alternative ATGs, were all found to be important quantitatively for activity. No other features were found to be of general significance, although the effects of secondary structure could be seen as outliers. A comparison to a large number of natural E. coli translation initiation sites showed the information profile to be qualitatively similar although differing quantitatively. This was probably due to the selection for good translation initiation sites in the natural set compared to the low average activity of the randomised set.

In a recent study, Bandmann and Nygren (2007) evaluated two combinatorial library strategies for their capability of tuning recombinant protein production in the cytoplasm of E. coli. Large expression vector libraries were constructed through either conservative (ExLib1) or free (ExLib2) randomisation of a seven-amino-acid window strategically located between a degenerated start codon and a sequence encoding a fluorescent-tagged target protein. Flow cytometric sorting and analyses of libraries, subpopulations or individual clones were followed by SDS-PAGE, western blotting, mass spectrometry and DNA sequencing analyses. For ExLib1, intracellular accumulation of soluble protein was shown to be affected by codon specific effects at some positions of the common N-terminal extension. Interestingly, for ExLib2 where the same sequence window was randomised via seven consecutive NN(G/T) tri-nucleotide repeats, high product levels (up to 24-fold higher than a reference clone) were associated with a preferential appearance of novel SD-like sequences. The method, promoters, vectors and host cells according to the invention, are neither disclosed nor suggested therein.

WO 01/98453 reports the isolation of optimised bacterial constructs by a survival assay applied to E. coli bacteria transformed with a library of trp promoters driving the expression of the TrpL-CAT fusion. Unexpected non-silent mutations in the TrpL part conferring levels of chloramphenicol resistance that are not observed with the wild-type TrpL sequence are described. Moreover, the clone traits seen with these mutants driving improved expression of the reporter TrpLₘᵤₜ-CAT fusion can be transposed to genes of interest fused to the optimised TrpL mutants. The method, promoters, vectors and host cells according to the invention, are neither disclosed nor suggested therein.

Therefore, the finding of a novel and powerful method for adapting, changing or optimising promoters in order to preferably drive high expression of therapeutic proteins would be extremely useful in the field of industrial production of therapeutic proteins.

### SUMMARY OF THE INVENTION

The present invention provides a method allowing the untranslated region (UTR) of the ribosome binding site (RBS) to be adapted to a given gene coding for a recombinant protein of interest, thus creating gene-specific mRNA secondary structures that maximize the efficiency of translation initiation.

A first aspect of the invention relates to a method for optimising the ribosome binding site of a promoter for the expression of a gene encoding a polypeptide of interest, placed under the control of said promoter, comprising the step of:
(a) preparing a screening cassette by fusing the 5′ end of the gene encoding a polypeptide of interest upstream of a reporter gene;
(b) cloning the screening cassette obtained in step (a) downstream of a library of mutagenised promoters generating thereby a library of mutagenised expression vectors;
(c) obtaining a library of clones by transforming host cells with the mutagenised vectors of step (b);
(d) culturing the library of clones of step (c) and selecting clones by monitoring the expression of the reporter gene with known methods.

A second aspect of the invention relates to the promoters obtained according to the method of the present invention.

A third aspect of the invention relates to the vectors comprising the promoters of the present invention.

The fourth aspect of the invention relates to a host cell transfected with at least one vector described above.

A fifth aspect of the invention relates to a method for producing a polypeptide of interest comprising the step of transfecting the host cell with a vector according to the invention.

### DETAILED DESCRIPTION OF THE INVENTION

Cloning a gene downstream of a bacterial promoter creates a new DNA sequence spanning the initiating ATG codon. This region, once transcribed into a messenger RNA, becomes the site of a sequential process involving recognition, of the mRNA in the ribosome binding site (RBS), by the small 30S ribosome subunit bound to initiation factors, positioning of the 30S subunit through a Shine-Dalgarno (SD)-anti-SD hydrogen bonding, interaction of the start codon with the initiator fMet-tRNA at the ribosomal P site and finally, recruitment of the large 50S particle joining the complex to form a complete ribosome (McCarthy and Brimacombe, 1994).

The present invention provides a method allowing the untranslated region (UTR) of the ribosome binding site (RBS) of a promoter to be adapted to a given gene of interest coding for a recombinant protein of interest, placed under the control of said promoter in a prokaryotic cell, thus creating gene-specific mRNA secondary structures that maximize the efficiency of translation initiation. According to the method of the invention, an intermediate screening cassette, wherein the 5′ end of the gene of interest is fused to a reporter gene, is cloned downstream of a library of promoters generating thereby a library of clones having a range of expression levels of the reporter gene. Depending on the expression level desired, clones are then selected and the promoters used for the expression of the protein of interest.

Therefore, a first aspect of the invention relates to a method for optimising the ribosome binding site of a promoter for the expression of a gene encoding a polypeptide of interest, placed under the control of said promoter, comprising the step of:
(a) preparing a screening cassette by fusing the 5′ end of the gene encoding a polypeptide of interest upstream of a reporter gene;
(b) cloning the screening cassette obtained in step (a) downstream of a library of mutagenised promoters generating thereby a library of mutagenised expression vectors;
(c) obtaining a library of clones by transforming host cells with the mutagenised vectors of step (b);
(d) culturing the library of clones of step (c) and selecting clones by monitoring the expression of the reporter gene with known methods.

Unless otherwise indicated, nucleic acids are written left to right in 5′ to 3′ orientation.

The “transcription start site” means the first nucleotide to be transcribed and is designated +1. Nucleotides downstream of the start site are numbered e.g. +2, +3, +4, and nucleotides in the opposite (upstream) direction are numbered e.g. −1, −2 or −3.

As used herein, the term “nucleic acid” includes RNA, DNA and cDNA molecules. The term nucleic acid is used interchangeably with the term “polynucleotide”. An “oligonucleotide” is a short chain nucleic acid molecule (about 2-60 nucleotides). A primer is an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced.

As used herein, the term “gene” means the segment of DNA involved in producing a polypeptide chain, that may or may not include regions preceding and following the coding region (e.g. 5′ untranslated (5′ UTR) or “leader” sequences and 3′ UTR or “trailer” sequences). Genes can be adapted to bacterial expression by codon usage-optimisation.

The term “promoter” as used herein refers to a region of DNA that functions to control the transcription of one or more DNA sequences, and that is structurally identified by the presence of a binding site for DNA-dependent RNA-polymerase and of other DNA sequences, which interact to regulate promoter function.

“Optimising the RBS” as used herein, means to introduce changes in the primary nucleic acid sequence and preferably to create gene-specific mRNA secondary structures in the RBS region. In this manner it is possible to increase or adapt the expression of the protein of interest by modulating the efficiency of translation initiation. Optimising the RBS may also be understood as adapting it to a desired level of expression of a polypeptide of interest.

As used herein, the term “ribosome binding site” or “RBS” is the sequence of the mRNA at which assembly of the 30S and 50S subunits of the ribosome takes place to initiate translation of an encoded protein. It generally extends from the −20 to the +13 position with respect to the +1 position of the first nucleotide of the initiation codon (Gold, 1988).

Promoters are well known in the art and include tac promoter, trc promoter, lac promoter, tac promoter (Ptac), trp promoter, lambda-PL promoter, aambda-PR promoter, lacUV5 promoter, araBAD promoter, lpp promoter, and a lpp-lac promoter. Other promoters known by the skilled person may be applied equally well in the invention.

A preferred promoter to be used in the frame of the method of the invention is the tac promoter. Ptac contains the −10 region of the lac promoter and the −35 region of the trp promoter; it is able to drive efficient expression upon IPTG (isopropylthio-β-D-galactoside) addition. This hybrid promoter made of Ptrp and Plac parts, described in the early 80s by Amann et al. (1983) and de Boer et al. (1983), is known to combine the strength of Ptrp with the regulation mode of Plac.

The “5′ end of the gene encoding a polypeptide of interest” refers to a segment of the polypeptide of interest coding sequence including the initiation codon. It refers to the first nucleic acids of the polypeptide of interest coding sequence.

In a preferred embodiment of the present invention, the 5′ end of the gene encoding the polypeptide of interest comprises the ribosome binding site or part thereof downstream of the +1 transcription start site. In further preferred embodiments, the 5′ end of the gene encoding a polypeptide of interest comprises at least the first +1 to +9, or +1 to +13, or +1 to +20, or +1 to +30, or +1 to +40, or +1 to +50, or +1 to +60, or +1 to +70, +1 to +80 or +1 to +150 nucleic acids encoding the polypeptide of interest. Most preferably, the 5′ end of the gene encoding the polypeptide of interest comprises the first +1 to 27 nucleic acids encoding the polypeptide of interest.

The 5′ end of the gene can correspond to the complete gene encoding the polypeptide of interest provided that the fusion with the reporter gene is soluble, active and functional allowing thereby the monitoring of the reporter gene by known methods.

A “screening cassette” or an “intermediate screening cassette” according to the invention, refers to a fusion between the 5′ end of the gene encoding a polypeptide of interest upstream of a reporter gene.

For the purpose of this invention “library of mutagenised promoters” or “library of promoters” refers to a population of artificial promoters. A library will preferably be derived from the same precursor promoter. The library of promoters can be generated by introducing mutations in the promoter by known mutagenesis methods such as, for example, by PCR methods or methods making use of physical-chemical agents (e.g. UV radiation, chemicals). In a preferred embodiment of the invention, the library is a modified ribosome binding site (RBS) library or an “RBS random library” created by introducing random mutations in the untranslated region of the ribosome binding”. More preferably, the mutations introduced in the RBS region are semi-random mutations wherein a purine-rich region corresponding to SD sequence is retained.

“A library of mutagenised expression vectors” refers to a population of expression vectors containing the screening cassette according to the invention.

A “library of clones” refers to a population of host cells grown under essentially the same growth conditions and which are identical in most of their genome but include a promoter or an expression vector library as defined herein. A library of bacterial clones will have different levels of expression of the same reporter gene (screening cassette).

As used herein, the terms “transforming” or “transfecting” used in reference to a cell means introducing or incorporated a non-native (heterologous) nucleic acid sequence into its genome or as an episomal plasmid that is maintained through two or more generations e.g. introducing a vector into the cell.

The term “reporter gene” or “reporter protein”, as used herein, is intended to mean a gene encoding a gene product that can be identified using simple, inexpensive methods or reagents, and that can be operably linked to a promoter or a gene encoding a polypeptide of interest or a portion thereof. Reporter genes may be used to determine transcriptional activity in screening assays (see, for example, Goeddel (ed.), Methods Enzymol., Vol. 185, San Diego. Academic Press, Inc. (1990)), e.g. using Luciferase as reporter gene (Wood, 1991; Seliger and McElroy, 1960; de Wet et al. (1985), or commercially available from Promega®).

Known reporter genes may be used according to the invention. Examples for reporter genes are selected from the group consisting of a bacterial β-galactosidase gene, a chloramphenicol acetyl transferase reporter gene, a β-lactamase gene, an alkaline phosphatase gene, a luciferase reporter gene, and a Green Fluorescent Protein gene. In a preferred embodiment, the reporter gene is a chloramphenicol acetyl transferase gene (CAT).

The polypeptide of interest, to which the method of the present invention can be applied, may be any polypeptide for which production is desired. The polypeptide of interest may find use in the field of pharmaceutics, agribusiness, drug discovery, structural biology or furniture for research laboratories. Preferred proteins of interests find use in the field of pharmaceuticals.

The polypeptide of interest may be, e.g., a naturally secreted protein, a normally cytoplasmic protein, a normally transmembrane protein. In preferred embodiments, the polypeptide of interest may be, e.g., a chemokine, growth factor, cytokine, hormone, antigen, receptor, antibody or any of its derivatives. As another example, the polypeptide of interest may be, e.g. a semi- or fully-synthetic molecule. The polypeptide of interest may be of any origin. Preferred polypeptides of interest are of human origin.

In preferred embodiments, the protein of interest is selected from the group consisting of insulin, growth hormone (GH), granulocyte macrophage colony-stimulating factor (GM-CSF), interferon 1a, interferon 2a, interferon 2b, interleukin-1, interleukin-11, interleukin 17F (IL-17F), interleukin-1 receptor antagonist, fibroblast growth factor 18 (FGF-18), chemokines (Rantes, MCP-1, SDF-1) or any molecules derived thereof.

As used herein, “host cell” refers to a cell that has the capacity to act as a host and expression vehicle for an introduced DNA (exogenous) sequence according to the invention. Any prokaryotic cell is suitable for performing the methods of the inventions. Examples of preferred bacterial host cells include Streptococci, Staphylococci, Streptomyces and Bacillus subtilis cells. More preferably, said cell is Escherichia coli (E. coli).

As used herein, the term “selecting” refers to the choice of some specific cells from a group of cells. In the frame of the present invention the selection of the cells is performed by assessing the transcriptional activity of the reporter gene in screening assays. If it is desired in the optimisation method according to the invention to increase the translation efficiency, then the best clones or high expressors are selected. If at the contrary it is aimed at obtaining a lower level of expression, then the clones that show the respective desired expression level are selected. In this manner the invention can provide clones exhibiting any level of expression of a protein of interest as desired. For example, when CAT reporter gene is used, clones over-expressing chloramphenicol acetyltransferase (CAT) or high expressors are screened in a survival assay based on resistance to the antibiotic chloramphenicol. If a lower expression is desired, a reporter protein that converts a chemical into a toxin can be used as only low expressors would be able to survive.

In a preferred embodiment, the method of the present invention further comprises the steps of replacing downstream of the modified promoters of the selected clones of step (d) the intermediate screening cassette of step (b) with the complete coding sequence of the gene of interest and producing the polypeptide of interest.

In a further preferred embodiment of the invention, the method relates to the optimisation of the ribosome binding site of a promoter for the expression of a gene encoding a polypeptide of interest, placed under the control of said promoter, comprising the step of:
(a) preparing a screening cassette by fusing the 5′ end of the gene encoding a polypeptide of interest upstream of a Chloramphenicol Acetyl Transferase (CAT) gene;
(b) cloning the screening cassette obtained in step (a) downstream of a library of mutagenised promoters created by the introduction of semi-random mutations in the UTR of the RBS generating thereby a library of mutagenised expression vectors;
(c) obtaining a library of clones by transforming host cells with the mutagenised vectors of step (b);
(d) culturing the library of clones of step (c) and selecting clones with the most efficient promoters in a survival assay based on resistance to increasing levels of chloramphenicol.

The method of the above further preferred embodiment may further comprise the steps of replacing downstream of the modified promoters of the selected clones of step (d) the intermediate screening cassette of step (a) with the complete coding sequence of the gene of interest and producing the polypeptide of interest.

In the examples given below, it has been shown that the replacement of the screening cassette downstream of the selected most efficient promoters with the complete sequence of a gene encoding a polypeptide of interest retains the high expression observed during the screening step (d) according to the method of the invention.

A second aspect of the invention relates to the promoters obtained according to the method of the present invention.

As shown in the examples below, the method of the invention was applied to the tac promoter of two constructs expressing either a cytokine (Interleukin17F-IL-17F) or a chemokine (Stromal Derived Factor 1α, SDF-1α) that was successfully optimised resulting in a strong expression of the desired proteins. In a preferred embodiment, the promoter obtained according to the method of the invention, is the tac promoter comprising at its 3′ end a nucleic acid sequence chosen from the sequences SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

A third aspect of the invention relates to the vectors or constructs comprising the promoters of the present invention.

As used herein, the term “vector” refers to a nucleic acid construct designed for transfer between different host cells. A vector may be a plasmid, a bacteriophage, a cloning vector, a shuttle vector or an expression vector. An “expression vector” refers to a vector that has the ability to incorporate and express heterologous DNA fragments in a foreign cell. Many prokaryotic and expression vectors are commercially available.

The fourth aspect of the invention relates to a host cell transfected with at least one vector described above.

A fifth aspect of the invention relates to a method for producing a polypeptide of interest comprising the step of transfecting the host cell with a vector according to the invention. The transfected host cell is cultured under conditions that cause the expression of the polypeptide of interest protein. The protein so produced can then be purified by techniques known to those skilled in the art and/or assayed for production by means consistent with the protein produced (e.g. Western blot, immunoassay, protein staining of a protein gel and/or enzymatic activity). Such purification and/or protein assay methodologies can also be employed to ascertain the level(s) of protein production.

In a preferred embodiment, the polypeptide of interest is IL-17F and the host cell is transfected with a vector comprising a tac promoter. Most preferably the tac promoter comprises at its 3′ end a nucleic acid sequence chosen from the sequences SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

In a further preferred embodiment, the polypeptide of interest is SDF-1α and the host cell is transfected with a vector comprising a tac promoter. Most preferably the tac promoter comprises at its 3′ end a nucleic acid sequence chosen from the sequences SEQ ID NO: 2, SEQ ID NO: 3 or SEQ ID NO: 4.

The polypeptide produced in accordance with the present invention may serve any purpose, and preferably it is a therapeutic protein intended for administration to humans or animals.

Depending on the intended use, the cell itself having the polypeptide integrated may be the product of the process according to the invention. Such a cell may e.g. be used for cell-based therapy.

Advantageously, the inventors could provide with the inventive method a simple and fast way to optimise the UTR of a promoter specifically for the expression of a gene of interest. In particular, with the method of the invention, the whole process from the construction and screening of the library of mutagenised promoter, to evaluation of clones expressing the full length protein of interest compared with the original wild type promoter construct takes no more than two to three months to complete.

Further aspects and advantages of the present invention will be disclosed in the following examples, which should be considered as illustrative only, and do not limit the scope of this application.

### BRIEF DESCRIPTION OF THE DRAWINGS

FIG. 1. SDS-PAGE gel showing SDF-1α and IL-17F expression under the tac (p404-Ptac) and T7 (pET30a) promoters. An arrow indicates the bands at the expected molecular weight for SDF-1α and IL-17F. (−) un-induced culture, (+) induced culture.

FIG. 2. Screening of SDF1′-CAT and IL17F′-CAT clones by CAT activity measurement:

FIG. 2A. CAT expression levels of 20 clones from the SDF1′-CAT library compared to the wild-type (WT) level obtained with p404-Ptac-SDF1′-CAT in E. coli DH10B.

FIG. 2B. CAT expression levels of 64 clones from the IL-17F′-CAT library compared to the wild-type (WT) level obtained with p404-Ptac-IL17F′-CAT in E. coli DH10B.

FIG. 3. SDS-PAGE gel showing SDF-1 expression levels under the optimised promoters of pTacS1, pTacS10 and pTacS13 and wild-type Ptac (p404-Ptac) promoter. An arrow indicates the band at the expected molecular weight for SDF-1α. (−) un-induced culture, (+) induced culture.

FIGS. 4A and 4B.

SDS-PAGE gels showing IL-17F expression levels under the optimised promoters of the pTacI series (#6, #17, #27, #37, #40, #43 and #53) and wild-type Ptac (p404-Ptac) promoter. An arrow indicates the band at the expected molecular weight for IL-17F. (−) un-induced culture, (+) induced culture.

FIG. 5A. SDS-PAGE gel showing IL-17F expression levels under the optimised promoters of pTacS1, pTacS10 and pTacS13 and wild-type Ptac (p404-Ptac) promoter. An arrow indicates the band at the expected molecular weight for IL-17F. (−) un-induced culture, (+) induced culture.

FIG. 5B. SDS-PAGE gel showing SDF-1α expression levels under the optimised promoters of pTacI6, pTacI7, pTacI17, pTacI27, pTacI 40, pTacI53 and wild-type Ptac (p404-Ptac) promoter. An arrow indicates the band at the expected molecular weight for SDF-1α.

### EXAMPLES

The E. coli BL21DE3 and W3110 strains were respectively purchased from Merck-Novagen (distributed by VWR International Life Science) and the American Type Culture Collection (ATCC, distributed in Europe by LGC Promochem).

The pET vectors used to generate T7-controlled expression vectors are from Merck-Novagen. The pFLAG-CTC vector carrying a mutated form of Ptac is from Sigma-Aldrich.

### Example 1

### Construction of the p404-tac Vector

Construction of the p404 Vector Framework

A derivative of the bacterial expression vector pET24a (Novagen) was generated by PCR using 2 oligonucleotide primers AS450 and AS451 (Table 1).

The resulting PCR product was digested with BamHI and circularised to create a 3555 bp promoter-less variant of pET24a which retained the bacterial origin of replication, f1 origin of replication and the kanamycin resistance gene together with a new multiple cloning site (MCS), containing recognition sites for NotI, NdeI, BamHI, XhoI, SacI and EcoRI. This plasmid was referred to as the p404 backbone. The complete sequence was confirmed using a set of custom sequencing primers AS537-AS541.

A transcription termination sequence identical to the rrnB-T1/T2 terminator present in vectors such as pMAL-2X (New England Bioloabs), was derived from an in-house bacterial expression plasmid (ID#16584). The sequence was amplified by PCR using the primers AS462 and AS463 each flanked at the 5′ end with the recognition sequence for EcoRI. The resulting fragment was digested with EcoRI, separated by electrophoresis through 0.8% agarose and purified using the Wizard DNA purification kit (Promega) according to the manufacturer's instructions. In parallel the p404 backbone was digested with EcoRI, dephosphorylated using calf intestinal alkaline phosphatase (Roche) according to the manufacturer's instructions and purified following gel electrophoresis as described above. The EcoRI-digested vector and rrnB DNAs were ligated together using the rapid DNA ligation kit (Roche) and aliquots of the ligation were used to transform the bacterial strain, JM101. Minipreps were prepared from individual colonies and the orientation of the rrnB transcriptional terminator was checked by sequence analysis. One vector with the desired orientation received the name p404-rrnB.

### Insertion of the Tac Promoter

The tac promoter is a hybrid sequence comprising the strong trp promoter followed by the lac operator region. Transcription is repressed by the binding of the lacI gene product to the lac operator and can be de-repressed by addition of IPTG. The tac promoter together with the adjacent lacI gene was amplified by PCR from the commercially available vector pFLAG-CTC (Sigma-Aldrich) using the two PCR primers ASMC008 and ASMC009. The two primers are flanked by the restriction enzyme recognition sites for NdeI and NotI respectively and were designed to restore the modified sequence present in the pFLAG-CTC to the original sequence described by Amman et al. (Table 1). The resulting 1300 bp fragment was digested with NotI and NdeI, purified following electrophoresis in 0.8% agarose and ligated into the corresponding NotI and NdeI restriction sites in the p404-rrnB construct described above. The resulting vector is called p404-Ptac.

TABLE 1
Oligonucleotides sequences (5′ → 3′ orientation)

| | | Sequence |
|---|---|---|
| Number | Sequence | number |
| AS450 | GCGGATCCCTCGAGCTCGAA | SEQ ID NO: 13 |
| | TTCTGGCGAATGGGACGCGC | |
| AS451 | GCGGATCCAACGTTCATATG | SEQ ID NO: 14 |
| | GCGGCCGCGCGCAACGCAAT | |
| | TAATGTAAG | |
| AS462 | GCGAATTCCCCATGCGAGAG | SEQ ID NO: 15 |
| | TAG | |
| AS463 | GCGAATTCTGTAGAAACGCA | SEQ ID NO: 16 |
| | AAAAGGC | |
| ASMC008 | AAGGCATATGCTGTTTCC | SEQ ID NO: 17 |
| | TGTGTGAAATTGTTATC | |
| ASMC009 | AAGGGCGGCCGCTTATCA | SEQ ID NO: 18 |
| | CTGCCCGCTTTCCAGTC | |
| AS537 | CAAATATGTATCCGCTCATG | SEQ ID NO: 19 |
| AS538 | GCTTGATGGTCGGAAGAGGC | SEQ ID NO: 20 |
| AS539 | CCAGTGGCTGCTGCCAGTGG | SEQ ID NO: 21 |
| AS540 | CATATATGGTGCACTCTCAG | SEQ ID NO: 22 |
| AS541 | CACTCAGGGTCAATGCCAGC | SEQ ID NO: 23 |
| ASMC030 | GGAACTCGAGTTACGCCCCGC | SEQ ID NO: 24 |
| ASMC033 | GGAACATATGAAACCGGTGAG | SEQ ID NO: 25 |
| | CCTGAGCTATCGTGAGAAAAA | |
| | AATCACTGGATATACC | |
| ASMC038 | GAAAACTGCAGGTCGAGGGGG | SEQ ID NO: 26 |
| | TCATGGCTG | |
| ASMC039(*) | GGGAATTCCATATGNNNNNYY | SEQ ID NO: 27 |
| | YYYYYNNNAATTGTTATCCGC | |
| | TCACAATTC | |
| ASMC040 | GGGAATTCCATATGCGTAAAA | SEQ ID NO: 28 |
| | TTCCGAAAGTTGGTCATGAGA | |
| | AAAAAATCACTGGATATACC | |

(*)Y stands for a mixture of T and C nucleotides

Cloning of hSDF-1α and hIL17F into p404-Ptac

To test the characteristics of p404-Ptac, the expression of two proteins hSDF-1α 1 and hIL17F was studied. The sequences encoding these two proteins have previously been optimised for expression in E. coli by deletion of the signal sequence and optimisation of the codon usage by GeneArt (Regensburg, Germany). The coding sequences were excised from existing in-house plasmids built on the pET30a template (Novagen) pET30a-SDF-1 alpha (#18496) and pET30a-IL17F (#18074) by digestion with NdeI and XhoI restriction endonucleases. The resulting DNA fragments were cloned into the corresponding sites of the p404-Ptac vector generating two expression plasmids that were called p404-Ptac-SDF1 (#18722) and p404-Ptac-IL17F (#18723).

The codon adapted sequences of human SDF-1α and IL-17F are reported herein as SEQ ID NO: 29 and as SEQ ID NO: 30 respectively.

### Evaluation of SDF-1α and IL-17F Expression Under the tac and T7 Promoters

E. coli W3110 bacteria were transformed with p404-Ptac-SDF1 or p404-Ptac-IL17F and allowed to grow in LB medium+kanamycin. As a control, the same experiment was performed with E. coli BL21DE3 (Novagen) transformed with pET30a-SDF-1 (#18496) or pET30a-IL17F (#18074) in which the respective genes are expressed under control of the T7 promoter. When the cell density reached an OD₆₀₀ₙₘ of 0.6, each culture was divided in two parts, one receiving 0.5 mM IPTG (“+”) and the other being kept as un-induced control (“−”). All cultures were further incubated for 3 hours. The resulting cell culture was centrifuged to recover a cell pellet. One aliquot (amount of cells corresponding to 1 ml at OD₆₀₀ₙₘ=1) was lysed in 200 μl BugBuster buffer+20 U/ml Benzonase (Novagen), denatured at 70° C. for 10 min and 10 μl were loaded on 10% MES SDS-PAGE gels (Invitrogen).

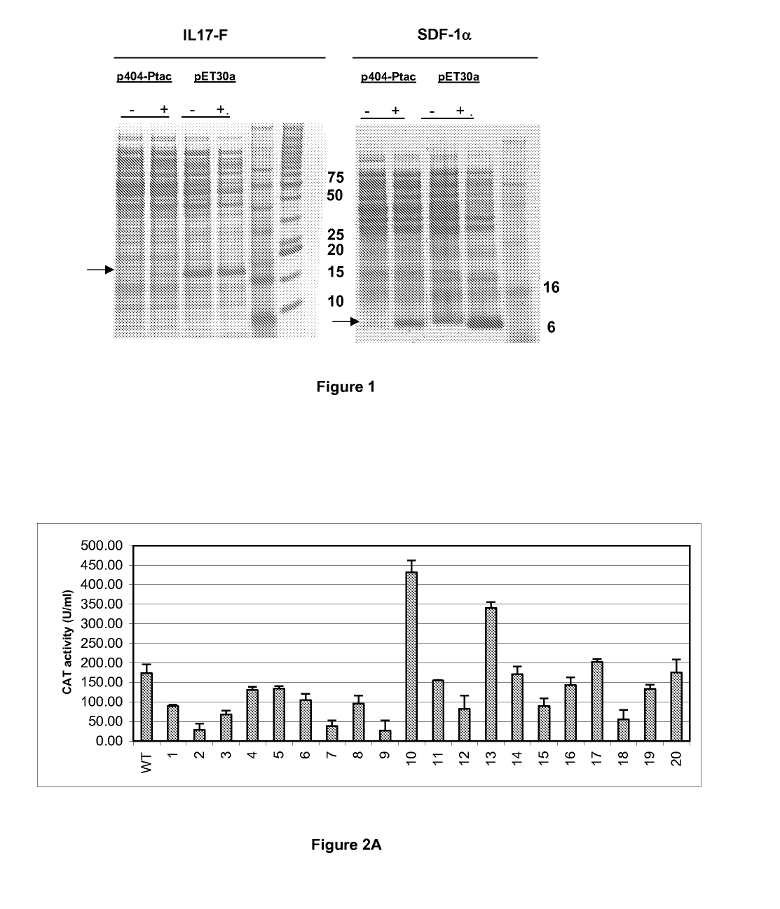

An arrow in FIG. 1 indicates the expected positions of SDF-1α and IL-17F protein bands.

### Example 2

### Optimisation of the Ptac Ribosome Binding Site (RBS)

Initial studies using p404-Ptac-SDF1 alpha and p404-Ptac-IL17F indicated only low-level expression of recombinant SDF-1α and IL-17F, as illustrated on FIG. 1. In order to improve expression levels, random mutations were introduced into the promoter RBS. As described above, the reasoning was that efficient expression may be influenced by mRNA secondary structure around the initiator methionine and thus should be optimised for each protein coding sequence to be expressed. To this end, p404-Ptac expression constructs were generated in which the first 27 nucleotides of each coding sequence were fused to the bacterial chloramphenicol acetyl transferase (CAT) gene. In this way promoter mutations resulting in increased expression could be screened directly by selecting for growth in increasing concentrations of chloramphenicol.

p404-Ptac/CAT Constructs

To generate fusion proteins, the CAT coding sequence was amplified by PCR using a forward primer in which the 5′ end of the CAT sequence was extended by an additional sequence consisting of the first 27 nucleotides of the SDF-1α or IL17F coding sequences. In both cases, upstream and downstream PCR primers were flanked by restriction sites for NdeI and XhoI respectively. Primers to generate SDF1′-CAT were ASMC033 (upstream) and ASMC030 (downstream); primers to generate IL17F′-CAT were ASMC040 (upstream) and ASMC030 (downstream). Amplified PCR products were digested with NdeI and XhoI and cloned into the corresponding sites of p404-Ptac to generate plasmid p404-Ptac-SDF1′-CAT and p404-Ptac-IL17F′-CAT.

### Construction of the RBS Random Library

In most E. coli mRNAs, the ribosome binding site (RBS) spans the initiator ATG. In the p404-Ptac vector, it was assumed that the RBS includes 20 nucleotides immediately upstream of the NdeI site (TCACACAGGAAACAGCATATG) and extends approximately 13 nucleotides into the protein coding sequence (Gold, 1988). The region upstream of the translation start site contains a purine rich element known as the Shine Dalgano (SD) sequence (bold type, underlined).

In order to optimise this region for protein expression, the mutagenic primer ASMC039 was used to introduce random mutations in the 15 nucleotides immediately upstream of the NdeI site, although retaining 7 purine residues in the SD core. For the PCR amplification, the mutagenesis primer was used together with the upstream primer ASMC038 (see Table 1 above). The PCR products thus obtained from this reaction were digested with PstI and NdeI and subjected to electrophoresis in a 0.8% agarose gel. A band of the predicted size of 0.6 kbp was purified using the Wizard purification system (Promega) and ligated into the corresponding sites of the parent vector expressing either SDF1′-CAT or IL17F′-CAT fusion proteins. In this way a library of recombinant plasmids was generated for each fusion protein. Each library were electroporated into ElectroMax E. coli DH10B (Invitrogen) and the library size was determined by plating serial dilutions on kanamycine-containing agar plates. The SDF1′-CAT and IL17F′-CAT libraries were shown to contain 6.7×10 exp 4 and 1.3×10 exp 5 clones per μl respectively.

### Screening of the RBS Random Library and Identification of Candidate Over-Expressors

An aliquot of each library was inoculated into 100 ml of fresh LB medium+kanamycin and allowed to grow overnight at 37° C. under agitation (200 rpm). The cultures were subsequently diluted to OD₆₀₀ of 0.3 in 100 ml of the same medium. After 3 hours of growth, 2×10 exp 4 bacteria from the SDF1′-CAT library and 2×10 exp 6 bacteria from the IL17F′-CAT library were spread on LB agar plates containing 600 mg/L chloramphenicol with or without 0.5 mM IPTG. Following overnight incubation of plates at 37° C., resistant clones appeared as colonies able to grow in the presence of this otherwise lethal dose of antibiotic. Based on their resistance characteristics, 20 clones from the SDF1′-CAT library and 64 from the IL17F′-CAT library were selected for further analyses. Control plates inoculated with DH10B cells carrying either p404-Ptac-SDF1′-CAT or p404-Ptac-IL17F′-CAT bacteria did not yield colonies after overnight incubation, thus showing that resistance to 600 mg/L chloramphenicol is not conferred by expression of the CAT fusion under the wild-type tac promoter.

All selected clones were grown in liquid medium and assayed for their intracellular CAT activity. The colonies were picked from agar plates and grown in Flat Bottom boxes (Qiagen) containing 1.3 ml of LB medium+kanamycin. Cultures were induced at OD₆₀₀=0.6 with 0.5 mM IPTG for 3 hours at 37° C. and 350 rpm. Cell pellets were lysed in BugBuster+Benzonase and the resulting cell extracts were analysed with the CAT-ELISA kit (Roche) according to the manufacturer's instructions. CAT levels were estimated against a calibration curve obtained with serial dilutions of a CAT standard and are expressed as arbitrary units. DH10B bacteria transformed with either p404-Ptac-SDF1′-CAT or p404-Ptac-IL17F′-CAT were included in the experiment and used as wild-type (WT) controls. Experiments were typically done in triplicate.

### Results

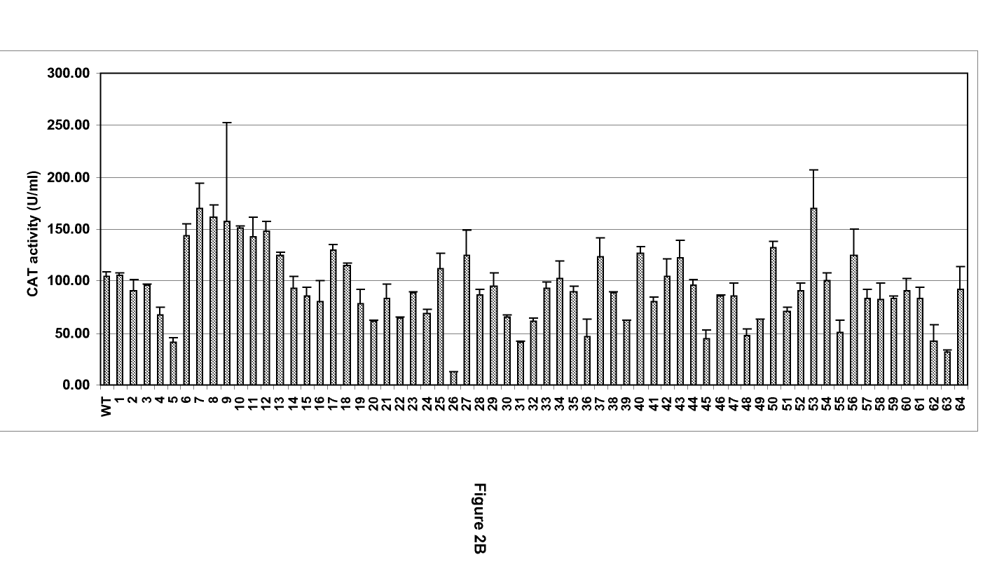

The data presented in FIG. 2A show that among the clones expressing SDF1′-CAT, three clones, referred to as pTacS1, pTacS10 and pTacS13, expressed a significantly higher CAT titre than the wild-type control. Of the 64 selected clones from the IL-17F′-CAT library, more than ten proved superior to the wild-type control (see FIG. 2B). Eight of these, designated pTacI6, pTacI7, pTacI17, pTacI27, pTacI37, pTacI40, pTacI43 and pTacI53, were selected for further analysis. Additional experiments confirmed the capacity of these clones to drive higher CAT expression than the wild-type control.

The sequences of the RBS region of all selected mutants were determined and are shown in Table 2. As expected, the 15 nucleotide sequence immediately upstream of the NdeI site is unique to each mutant. An additional C→T mutation 20 nucleotides upstream of the NdeI site is also present in all mutants, with the exception of pTacS1 and pTacI7, outside the region that was subjected to mutagenesis. pTacS1 has an additional G→A mutation 26 nucleotides upstream of the NdeI site.

TABLE 2
Sequence of the 3′ end of Ptac in 3 clones
isolated from the SDF1′-CAT and 8 clones
isolated from IL17F′-CAT libraries upstream
of the ATG initiating codon.

| Clone Name | Sequence | Sequence number |
|---|---|---|
| WT Ptac | GAGCGGATAACAATTTC | SEQ ID NO: 1 |
| | ACACAGGAAACAGCAT | |
| pTacS1 | GAGCAGATAACAATTTT | SEQ ID NO: 2 |
| | CGGAAAGAAGGTACAT | |
| pTacS10 | GAGCGGATAATAATTCA | SEQ ID NO: 3 |
| | AGAGGGAAAATCACAT | |
| pTacS13 | GAGCGGATAATAATTGC | SEQ ID NO: 4 |
| | CAAAGGAGTCCTTCAT | |
| pTacI6 | GAGCGGATAATAATTAT | SEQ ID NO: 5 |
| | GGAGAAAAGCAACCAT | |
| PtacI7 | GAGCGGATAACAATTCG | SEQ ID NO: 6 |
| | AGAAAAGAGTTCACAT | |
| pTacI17 | GAGCGGATAATAATTGC | SEQ ID NO: 7 |
| | CAAAGAGACACGACAT | |
| pTacI27 | GAGCGGATAATAATTCA | SEQ ID NO: 8 |
| | CGAAAGGACATATCAT | |
| pTacI37 | GAGCGGATAATAATTCA | SEQ ID NO: 9 |
| | CGAAAGGACATATCAT | |
| pTacI40 | GAGCGGATAATAATTCT | SEQ ID NO: 10 |
| | AGAAAGGAACCCCCAT | |
| pTacI43 | GAGCGGATAATAATTGC | SEQ ID NO: 11 |
| | TAGAGGAACTCTCCAT | |
| pTacI53 | GAGCGGATAATAATTTA | SEQ ID NO: 12 |
| | TGAAGGAGGCCCGCAT | |

The Ptac region that was submitted to mutagenesis is in underlined style. The purine rich element upstream of the translation start site is in bold and underlined style.

### Example 3

### SDF-1α and IL-17F Expression Levels Under the Optimised tac Promoters

The full length optimised SDF-1 coding sequence described above was amplified as an NdeI-XhoI DNA fragment and cloned into the corresponding sites of the three vectors from the pTacS series, generating three expression plasmids named pTacS1-SDF1, pTacS10-SDF1 and pTacS13-SDF1. In the same way, the NdeI-XhoI DNA fragment carrying the optimised IL-17F coding sequence was cloned into the corresponding sites of the seven vectors from the pTacI series, generating 7 expression plasmids named pTacI6-IL17F, pTacI17-IL17F, pTacI27-IL17F, pTacI37-IL17F, pTacI40-IL17F, pTacI43-IL17F and pTacI53-IL17F.

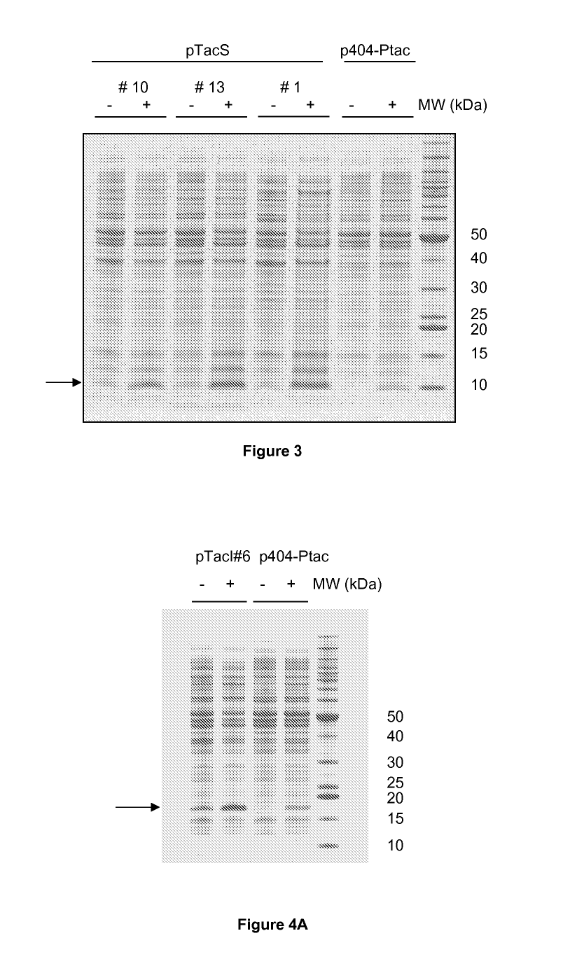

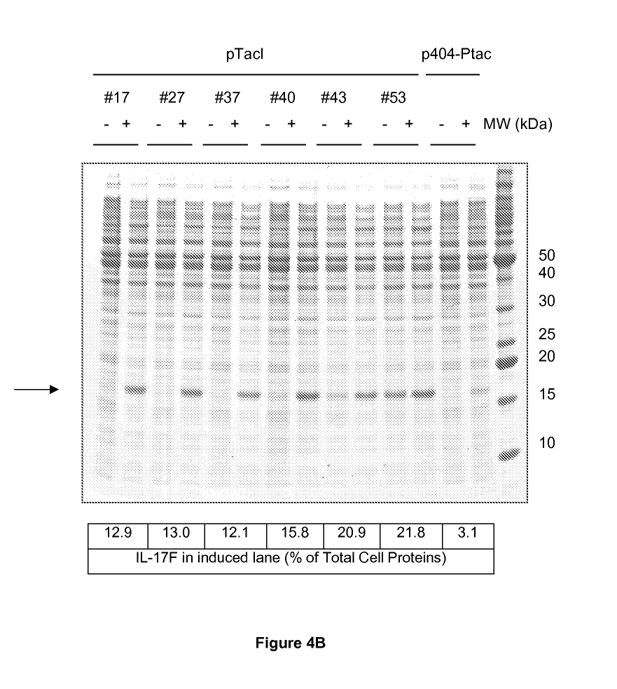

After transformation of each of these constructs into E. coli W3110, the bacteria were allowed to grow in LB medium+kanamycin. As a control, the same experiment was performed with E. coli W3110 transformed with p404-Ptac-SDF1 or p404-Ptac-IL17F in which the respective genes are expressed under control of the wild-type Ptac. When the cell density reached an OD₆₀₀ₙₘ of 0.6, each culture was divided in two parts, one receiving 0.5 mM IPTG (“+”) and the other being kept as un-induced control (“−”). All cultures were further incubated for 3 hours. The resulting cell culture was centrifuged to recover a cell pellet. One aliquot (amount of cells corresponding to 1 ml at OD₆₀₀ₙₘ=1) was lysed in 200 μl BugBuster buffer+20 μml Benzonase (Novagen), denatured at 70° C. for 10 min and 10 μl were loaded on 10% MES SDS-PAGE gels (Invitrogen). The recombinant protein levels obtained were estimated by SDS-PAGE analysis of total cell extracts (see FIG. 3 for SDF1 and FIGS. 4A and 4B for IL-17F).

### Results

The expected positions of SDF-1α and IL-17F protein bands are indicated by an arrow in FIGS. 3, 4A and 4B.

The gel in FIG. 3 shows a protein at the expected molecular weight for SDF-1α which has a stronger intensity in the samples from pTacS clones than from the original vector, both in un-induced and induced conditions. The higher un-induced level in pTacS clones can be explained by a more efficient translation of pre-induced mRNAs.

In FIGS. 4A and 4B, the almost complete absence of IL-17F expression under the original tac promoter is confirmed as already seen on FIG. 1. However, all induced extracts from the pTacI series display a strong band at the expected molecular weight for IL-17F (17 kDa). The level of expression of each clone was estimated by densitometry scanning of the Coomassie blue-stained SDS-PAGE gel using a BioRad GS800 densitometry scanner. In each induced sample, the intensity of all detectable protein bands b₁ to bₙ was converted into peak volumes PV, by the Quantity One software and the amount of IL-17F was expressed as a percentage of Total Cell Proteins (TCP) by the formula:

IL-17F=PV_{IL-17F}/Σ_{i=1->n}(PVᵢ)

With this method, it was demonstrated that the IL-17F protein amount varied between 12% and 22% of Total Cell Proteins in six pTacI clones, compared to 3% for the original tac promoter. Among the four clones that showed no detectable level of IL-17F in un-induced conditions (namely clones #17, 27, 37 and 40), pTacI40 gave the highest yield at 15.8% of TCP. The two last clones #43 and #53 displayed a significant expression leakage, as shown by the presence of a band at 17 kDa in their non-induced samples. This was probably a consequence of the optimisation strategy, which, by selecting optimised UTRs, identified clones with easily translatable mRNAs, whether they were transcribed in the presence or absence of the inducer. Between the two “leaky” clones, pTacI43 looked superior in that it led to a high-induced IL-17F level (21% of TCP), together with a minor level in un-induced conditions.

Taken together, these results demonstrated that using a survival assay designed to select for SDF1′-CAT and IL17F′-CAT overexpressors successfully led to optimised UTRs that, in turn, drove stronger expression of the full-length SDF-1α and IL-17F genes.

### Example 4

### Analysis of SDF-1α and IL-17F Expression Levels

To estimate the recombinant protein product titres obtained with the newly isolated clones, SDF-1α and IL-17F proteins were extracted, purified and quantified from cultures expressing the corresponding genes from plasmids pTacS10-SDF1, pTacI40-IL17F and pTacI43-IL17F and compared to the wild-type p404-Ptac-SDF1 and p404-Ptac-IL17F constructs as appropriate.

Briefly, the vectors were separately transformed into chemically competent E. coli W3110 bacteria and individual kanamycin resistant colonies were transferred into liquid LB medium in the presence of kanamycin. After incubation for 8 hours with continuous agitation at 37° C., the cultures were used to inoculate a 5-L fermenter containing sterile medium composed of 110 g/L glycerol, 5 g/L (NH₄)₂SO₄, Yeast Extract, phosphate buffer, calcium chloride, magnesium sulfate, trisodium citrate, salts, antifoam PPG2000 (Sigma) and 40 mg/L kanamycin sulfate. The operating parameters were T=37° C., oxygen tension maintained at 30% of air saturation by airflow, oxygen flow and stirrer controllers, and pH=7 was maintained by automatic injection of liquid ammonia. The cells were allowed to grow to an Optical Density (OD₆₀₀ₙₘ) of 30, and then induced for 3 to 5 hours with 1 mM IPTG. The resulting cell cultures were harvested by centrifugation and the cell pellet was either processed immediately or stored at −20° C.

Both recombinant proteins were found as inclusion bodies in the E. coli cytoplasm. To recover recombinant protein, cells were washed and broken with a high-pressure homogeniser. Inclusion bodies were recovered by centrifugation, washed, then solubilised in 6M guanidium chloride+DTT at 60° C. prior to buffer exchange into 8M urea+DTT.

The SDF-1α protein was then captured on a Fractogel SE-Hicap (Merck) cation exchange gel under reducing conditions and eluted in a single conductivity step. Then, the eluate was concentrated and refolded overnight by drop wise dilution into a buffer containing 0.1 M Tris-HCl+0.01 mM GSSG (oxidized glutathione)/0.1 mM GSH (reduced glutathione)+0.15 M arginine pH 8.5. The protein in solution was then concentrated by cation exchange chromatography (SP Sepharose Fast Flow, GE Healthcare). The buffer was exchanged to 0.1 M Tris-HCl pH 7.5 and the solubilised SDF-1α was submitted to Methionine Amino Peptidase (BTG Israel) digestion to remove the extra N-terminal methionine residue. A final affinity chromatography on Heparin-Sepharose Fast Flow (GE Healthcare) was applied as a ‘polishing’ step, mainly to remove aggregates and the Methionine Amino Peptidase enzyme. The purified protein was finally diafiltered in 50 mM Na-acetate+0.1 M NaCl pH 5 and concentrated to 1 mg/ml.

The IL-17F protein was partially purified through a single Fractogel EMD TMAE Hicap (Merck) cation exchange step and eluted in a single conductivity step.

Purified or semi-purified proteins were loaded on SDS-PAGE gels to check for purity. They were quantified by both UV measurements at 280 nm—assuming theoretical extinction coefficients of 8730 M⁻¹·cm⁻¹ and 14660 M⁻¹·cm⁻¹ for SDF-1α and IL-17F respectively—and by the BCA method (Pierce).

### Results

The data presented in Table 3 showed that, in the case of SDF-1α, the optimised tac promoter in the pTacS10 vector resulted in the production of 22 times more protein than the wild-type tac promoter. For IL-17F, the specific titre was increased by 10.3- and 11.5-fold using the optimised tac promoters in the vectors pTacI40- and pTacI43 respectively.

TABLE 3
SDF-1 and IL-17F expression titres obtained with wild-type and optimised tac
promoters.
The yields are expressed as mg of pure (SDF-1α) or semi-purified (IL-17F)
protein per gram of wet E. coli cell biomass.

| | SDF-1 | | | IL-17F | | |
|---|---|---|---|---|---|---|
| | p404-Ptac- | pTacS10- | | p404-Ptac- | pTacl40- | pTacl43- |
| | SDF1 | SDF1 (*) | | IL17F | IL17F | IL17F |
| mg protein/g wet cell paste | 0.1 | 2.4 | 2.2 | 1.8 | 22.7 | 26 |
| (UV) | | | | | | |
| mg protein/g wet cell paste | 0.1 | 2.1 | 2.1 | 3 | 24 | 26.1 |
| (BCA) | | | | | | |

| | | | | | |
|---|---|---|---|---|---|
| Improvement factor (UV) (**) | — | 23.0 | — | 12.6 | 14.4 |
| Improvement factor (BCA) (**) | — | 21.0 | — | 8.0 | 8.7 |

(*) Two cell pellets from the pTacS10-SDF1 construct were processed independently, giving two measurements for the same construct.
(**) Improvement factor calculated as the ratio between the protein yields obtained with the optimised construct (pTacS or pTacl) and the wild-type construct (p404), measured either by UV or by the BCA assay.

### Example 5

### Swapping Experiments

The strategy described in the invention generated two series of promoters optimised either for SDF-1α or IL-17F expression in E. coli. In order to test the efficency of these promoters in driving expression of other genes, an experiment was conducted where the SDF-1α and IL-17F encoding genes were exchanged and respectively introduced into vectors from the pTacI and pTacS series. Cloning of the SDF-1α and IL-17F genes as NdeI-XhoI DNA fragments into pTacI and pTacS vectors created so-called “swap vectors” that were evaluated under similar conditions as already described in Example 3.

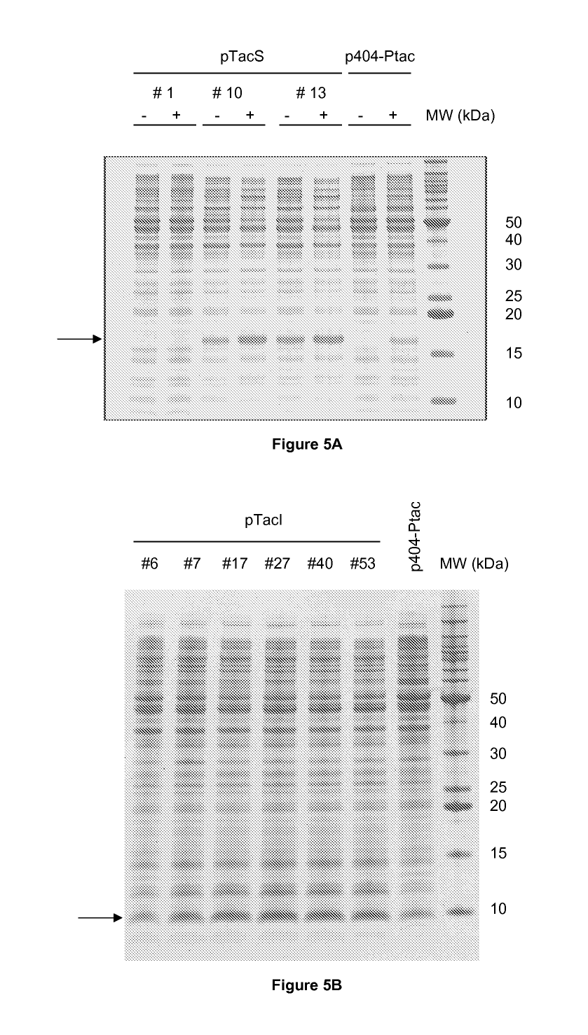

The gel in FIG. 5A shows a protein at the expected molecular weight for IL-17F which has a stronger intensity in the samples from two out of three pTacS clones—namely pTacS10 and pTacS13—than from the original vector, both in un-induced and induced conditions. Conversely, the level of SDF-1α expression demonstrated by the intensity of the corresponding band on the gel in FIG. 5B is found higher in five out the six vectors tested (pTacI7, pTacI17, pTacI27, pTacI40 and pTacI53) than from the original vector.

These experiments with “swap vectors” demonstrate the capacity of some of the promoters disclosed in the invention to act as universal promoters able to translate several genes more efficiently than the wild-type counterpart.

### REFERENCES

Amann, E., Brosius, J., and Ptnashe, M. (1983) Vectors bearing a hybrid trp-lac promoter useful for regulated expression of cloned genes in Escherichia coli. Gene 25: 167-178.
Bandmann, N., and Nygren, P. A. (2007) Combinatorial expression vector engineering for tuning of recombinant protein production in Escherichia coli. Nucleic Acids Res 35: e32.
Barrick, D., Villanueba, K., Childs, J., Kalil, R., Schneider, T. D., Lawrence, C. E. et al. (1994) Quantitative analysis of ribosome binding sites in E. coli. Nucleic Acids Res 22: 1287-1295.
Blattner, F. R., Plunkettlll, G., Bloch, C. A., Perna, N. T., Burland, V., Riley, M. et al. (1997) The Complete Genome Sequence of Escherichia coli K-12. Science 277(5): 1453-1462.
Boni, I. V., Isaeva, D. M., Musychenko, M. L., and Tzareva, N. V. (1991) Ribosome-messenger recognition: mRNA target sites for ribosomal protein S1. Nucleic Acids Res 19: 155-162.
Brock, J. E., Paz, R. L., Cottle, P., and Janssen, G. R. (2007) Naturally occurring adenines within mRNA coding sequences affect ribosome binding and expression in Escherichia coli. J Bacteriol 189: 501-510.
Chen, H., Bjerknes, M., Kumar, R., and Jay, E. (1994a) Determination of the optimal aligned spacing between the Shine-Dalgarno sequence and the translation initiation codon of Escherichia coli mRNAs. Nucleic Acids Research 22: 4953-4957.
Chen, H., Pomeroy-Cloney, L., Bjerknes, M., Tam, J., and Jay, E. (1994b) The influence of adenine-rich motifs in the 3′ portion of the ribosome binding site on human IFN-gamma gene expression in Escherichia coli. Journal of Molecular Biology 240: 20-27.
de Boer, H. A., Comstock, L. J., and Vasser, M. (1983) The tac promoter: a functional hybrid derived from the trp and lac promoters. Proc Natl Acad Sci USA 80: 21-25.
De Smit, M. H., and van Duin, J. (1994) Control of translation by mRNA secondary structure in Escherichia coli. A quantitative analysis of literature data. Journal of Molecular Biology 244: 144-150.
De Smit, M. H., and van Duin, J. (1990) Secondary structure of the ribosome binding site determines translational efficiency: a quantitative analysis. Proceedings of the National Academy of Sciences, USA 87: 7668-7672.
Gold, L. (1988) Posttranscriptional regulatory mechanisms in Escherichia coli. Annual Revue of Biochemistry 57: 199-233.
Graumann, K., and Premstaller, A. (2006) Manufacturing of recombinant therapeutic proteins in microbial systems. Biotechnol J 1: 164-186.
Komarova, A. V., Tchufistova, L. S., Dreyfus, M., and Boni, I. V. (2005) AU-rich sequences within 5′ untranslated leaders enhance translation and stabilize mRNA in Escherichia coli. J Bacteriol 187: 1344-1349.
Martin-Farmer, J., and Janssen, G. R. (1999) A downstream CA repeat sequence increases translation from leadered and unleadered mRNA in Escherichia coli. Mol Microbiol 31: 1025-1038.
McCarthy, J. E. G., and Brimacombe, R. (1994) Prokaryotic translation: the interactive pathway leading to initiation. Trends in Genetics 10: 402-407.
Mori, K., Saito, R., Kikuchi, S., and Tomita, M. (2006) Inferring rules of Escherichia coli translational efficiency using an artificial neural network. Biosystems.
Peti, W., and Page, R. (2007) Strategies to maximize heterologous protein expression in Escherichia coli with minimal cost. Protein Expr Purif 51: 1-10.
Ringquist, S., Schinedling, S., Barrick, D., Green, L., Binkley, J., Stormo, G. D., and Gold, L. (1992) Translation initiation in Escherichia coli: sequences within the ribosome-binding site. Molecular Microbiology 6: 1219-1229.
Shine, J., and Dalgarno, L. (1974) The 3′-terminal sequence of Escherichia coli 16S ribosomal RNA: complementarity to nonsense triplets and ribosome binding sites. Proc Natl Acad Sci USA 71: 1342-1346.
Terpe, K. (2006) Overview of bacterial expression systems for heterologous protein production: from molecular and biochemical fundamentals to commercial systems. Appl Microbiol Biotechnol 72: 211-222.
Tzareva, N. V., Makhno, V. I., and Boni, I. V. (1994) Ribosome-messenger recognition in the absence of the Shine-Dalgarno interactions. FEBS Lett 337: 189-194.

1-17. (canceled)

18. A method for optimising the ribosome binding site of a promoter for the expression of a gene encoding a polypeptide of interest, placed under the control of said promoter, comprising the steps of:
(a) preparing a screening cassette by fusing the 5′ end of the gene encoding a polypeptide of interest upstream of a reporter gene;
(b) cloning the screening cassette obtained in step (a) downstream of a library of mutagenised promoters generating thereby a library of mutagenised expression vectors;
(c) obtaining a library of clones by transforming host cells with the mutagenised vectors of step (b); and
(d) culturing the library of clones of step (c) and selecting clones by monitoring the expression of the reporter gene with known methods.

19. The method according to claim 18, further comprising the steps of replacing downstream of the modified promoters of the selected clones of step (d) the intermediate screening cassette of step (b) with the complete coding sequence of the gene of interest and producing the polypeptide of interest.

20. The method according to claim 18, wherein the 5′ end of the gene encoding a polypeptide of interest comprises the ribosome binding site or part thereof downstream of the +1 transcription start site.

21. The method according to claim 18, wherein the 5′ end of the gene encoding a polypeptide of interest comprises at least the first 1 to 150 nucleic acids encoding the polypeptide of interest.

22. The method according to claim 18, wherein the 5′ end of the gene encoding a polypeptide of interest corresponds to at least the first 10 to 50 nucleic acids encoding the polypeptide of interest.

23. The method according to claim 18, wherein the 5′ end of the gene encoding a polypeptide of interest corresponds to at least the first 27 nucleic acids encoding the polypeptide of interest.

24. The method according to claim 18, wherein the library of mutagenised promoters is created by introducing semi-random mutations in the untranslated region of the promoter's ribosome binding site.

25. The method according to claim 18, wherein the promoter is selected from the group consisting of a tac promoter, a trc promoter, a lac promoter, a Ptac, a trp promoter, a lambda-PL promoter, a lambda-PR promoter, a lacUV5 promoter, an araBAD promoter, a lpp promoter, and a lpp-lac promoter.

26. The method according to claim 18, wherein the reporter gene is selected from the group consisting of a bacterial β-galactosidase gene, a chloramphenicol acetyl transferase reporter gene, a β-lactamase gene, an alkaline phosphatase gene, a luciferase reporter gene, and a Green Fluorescent Protein gene.

27. The method according to claim 18, wherein the polypeptide of interest is selected from the group consisting of a chemokine, growth factor, cytokine, hormone, antigen, receptor, and an antibody or any of its fragments.

28. A promoter obtained by the method of claim 18.

29. The promoter according to claim 28, wherein said promoter is a Ptac promoter comprising at its 3′ end a nucleic acid sequence selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

30. A vector comprising:
a) a promoter according to claim 28; or
b) a Ptac promoter comprising at its 3′ end a nucleic acid sequence selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

31. A host cell transfected with a vector according to claim 30.

32. The host cell according to claim 31, wherein said cell is a prokaryotic host cell.

33. The host cell according to claim 32, wherein the host cell is Escherichia coli.

34. A method for producing a polypeptide of interest comprising culturing a host cell according to claim 31 under conditions suitable for the production of a polypeptide of interest.


SEQUENCE LISTING (30 sequences)

SEQ ID NO: 1 - DNA, 33 bp, Escherichia coli
  misc_feature (1)..(33): 3′ end of E. coli Tac promoter
gagcggataacaatttcacacaggaaacagcat

SEQ ID NO: 2 - DNA, 33 bp, Artificial sequence
  3′ end of E. coli promoter tac mutated region (PTacS1)
gagcagataacaattttcggaaagaaggtacat

SEQ ID NO: 3 - DNA, 33 bp, Artificial Sequence
  3′ end of E. coli promoter tac mutated region (PTacS10)
gagcggataataattcaagagggaaaatcacat

SEQ ID NO: 4 - DNA, 33 bp, Artificial Sequence
  3′ end of E. coli promoter tac mutated region (PTacS13)
gagcggataataattgccaaaggagtccttcat

SEQ ID NO: 5 - DNA, 33 bp, Artificial Sequence
  3′ end of E. coli promoter tac mutated region (PTacI6)
gagcggataataattatggagaaaagcaaccat

SEQ ID NO: 6 - DNA, 33 bp, Artificial Sequence
  3′ end of E. coli promoter tac mutated region (PTacI7)
gagcggataacaattcgagaaaagagttcacat

SEQ ID NO: 7 - DNA, 33 bp, Artificial Sequence
  3′ end of E. coli promoter tac mutated region (PTacI17)
gagcggataataattgccaaagagacacgacat

SEQ ID NO: 8 - DNA, 33 bp, Artificial Sequence
  3′ end of E. coli promoter tac mutated region (PTacI27)
gagcggataataattcacgaaaggacatatcat

SEQ ID NO: 9 - DNA, 33 bp, Artificial Sequence; identical to SEQ ID NO: 8
  3′ end of E. coli promoter tac mutated region (PTacI37)

SEQ ID NO: 10 - DNA, 33 bp, Artificial Sequence
  3′ end of E. coli promoter tac mutated region (PTacI40)
gagcggataataattctagaaaggaacccccat

SEQ ID NO: 11 - DNA, 33 bp, Artificial Sequence
  3′ end of E. coli promoter tac mutated region (PTacI43)
gagcggataataattgctagaggaactctccat

SEQ ID NO: 12 - DNA, 33 bp, Artificial Sequence
  3′ end of E. coli promoter tac mutated region (PTacI53)
gagcggataataatttatgaaggaggcccgcat

SEQ ID NO: 13 - DNA, 40 bp, Artificial Sequence
  Oligonucleotide primer AS450
gcggatccctcgagctcgaattctggcgaatgggacgcgc

SEQ ID NO: 14 - DNA, 48 bp, Artificial Sequence
  Primer 451
cggatccaacgttcatatggcggccgcgcgcaacgcaattaatgtaag

SEQ ID NO: 15 - DNA, 23 bp, Artificial Sequence
  Primer AS462
gcgaattccccatgcgagagtag

SEQ ID NO: 16 - DNA, 27 bp, Artificial Sequence
  Primer AS463
gcgaattctgtagaaacgcaaaaaggc

SEQ ID NO: 17 - DNA, 35 bp, Artificial Sequence
  Primer ASMC008
aaggcatatgctgtttcctgtgtgaaattgttatc

SEQ ID NO: 18 - DNA, 35 bp, Artificial Sequence
  Primer ASM009
aagggcggccgcttatcactgcccgctttccagtc

SEQ ID NO: 19 - DNA, 20 bp, Artificial Sequence
  Primer AS537
caaatatgtatccgctcatg

SEQ ID NO: 20 - DNA, 20 bp, Artificial Sequence
  Primer AS538
gcttgatggtcggaagaggc

SEQ ID NO: 21 - DNA, 20 bp, Artificial Sequence
  Primer AS539
ccagtggctgctgccagtgg

SEQ ID NO: 22 - DNA, 20 bp, Artificial Sequence
  Primer AS540
catatatggtgcactctcag

SEQ ID NO: 23 - DNA, 20 bp, Artificial Sequence
  Primer AS541
cactcagggtcaatgccagc

SEQ ID NO: 24 - DNA, 21 bp, Artificial Sequence
  Primer ASMC030
ggaactcgagttacgccccgc

SEQ ID NO: 25 - DNA, 58 bp, Artificial Sequence
  Primer ASMC033
ggaacatatgaaaccggtgagcctgagctatcgtgagaaaaaaatcactggatatacc

SEQ ID NO: 26 - DNA, 30 bp, Artificial Sequence
  Primer ASMC038
gaaaactgcaggtcgagggggtcatggctg

SEQ ID NO: 27 - DNA, 51 bp, Artificial Sequence
  Mutagenic primer
gggaattccatatgnnnnnyyyyyyynnnaattgttatccgctcacaattc

SEQ ID NO: 28 - DNA, 62 bp, Artificial Sequence
  Primer ASMC040
gggaattccatatgcgtaaaattccgaaagttggtcatgagaaaaaaatcactggatatacc

SEQ ID NO: 29 - DNA, 207 bp, Artificial Sequence
  human SDF-1a  coding sequence adapted for expression in E. Coli
atgaaaccggtgagcctgagctatcgttgcccgtgccgtttttttgaaagccatgtggcccgtgccaacgttaaacatctgaaaattctgaacaccccgaactgtgcgctgcagattgtggcccgtctgaaaaacaacaaccgccaggtgtgtattgatccgaaactgaaatggattcaggaatacctggaaaaagcgctgaacaaa

SEQ ID NO: 30 - DNA, 402 bp, Artificial Sequence
  Human IL-17F coding sequence adapted for expression in E. Coli
atgcgtaaaattccgaaagttggtcatacctttttccagaaaccggaaagctgtccgccggttccgggcggtagcatgaaactggatatcggcatcatcaacgaaaatcagcgtgtgagcatgagccgtaacattgaaagccgtagcaccagcccgtggaattataccgttacctgggatccgaatcgttatccgagcgaagttgttcaggcccagtgtcgtaacctgggctgtattaacgcccagggcaaagaggatatcagcatgaacagcgttccgattcagcaggaaaccctggtggttcgtcgtaaacatcagggttgtagcgtgagctttcagctggaaaaagtgctggttaccgttggttgtacctgtgtgaccccggtgattcatcacgttcag